(19) United States
(12) Patent Application Publication
(10) Pub. No.: US 2017/0369414 A1
(43) Pub. Date: Dec. 28, 2017

(54) O-Quinone Compounds as Agents Neutralising Nitric Oxide

(71) Applicant: Institut des Substances Végétales, Beaumont (FR)

(72) Inventors: Daniel Jean, Vic-Le-Comte (FR); Maryse Pouligon, Cournon-d'Auvergne (FR)

(73) Assignee: Institut des Substances Végétales, Beaumont (FR)

(21) Appl. No.: 15/541,790

(22) PCT Filed: Jan. 12, 2015
(86) PCT No.: PCT/FR2015/050064

§ 371 Date: Jul. 6, 2017

(51) Int. Cl. C07C 59/84; A61K 9/00; A61K 8/36; A61K 31/192; A61Q 19/00; A61K 36/68

(52) U.S. Cl. CPC C07C 59/84; A61Q 19/00; A61K 36/68; A61K 8/36; A61K 31/192; A61K 9/0053; A61K 2800/10

(57) ABSTRACT

The invention relates to O-quinone compounds of general formula (I) as agents neutralising nitric oxide, and to the therapeutic or cosmetic use thereof.

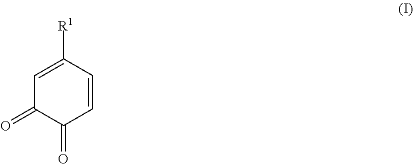

The present invention relates to the field of treating inflammatory diseases and/or disorders resulting from an excess of nitric oxide (NO); more particularly the object of the invention are o-quinone compounds as agents neutralizing nitric oxide and their therapeutic or cosmetic use.

Nitric oxide (NO) is one of the smallest molecules produced by biological systems. Its actions are also diverse in bodies as the cells capable of producing it. Indeed, its status of a relatively unstable free radical gives it its electronic structure (its half-life is 5 seconds) which allows it to act on a large number of molecules which are more or less sensitive to its action within cellular and extra-cellular metabolic processes.

In the 80s it was shown that nitric oxide specifically acted as an intermediate of vascular dynamics by controlling the relaxation of vessels. Among other cells, during inflammatory processes, this molecule is produced by endothelial cells (1,2) and macrophages (3,4) by enzymes, nitric oxide synthases (NOs), catalyzing the transformation of arginine into citrulline. These enzymes exist constitutively or are induced during immune reactions, these are referred to as iNOs. Vasodilation is induced via the stimulation of the guanylate cyclase of the smooth muscle (5). Nitric oxide is also formed by endothelial cells in response to a variety of substances like bradykinin (2), histamine and 5-hydroxytryptamine (6). Nitric oxide may also be formed by the macrophages activated by lipopolysaccharides or cytokines (6).

Nitric oxide also plays a role in immunity by its strong chemical reactivity which allows it to participate in the lysis of germs absorbed by the macrophages. In the presence of the super-oxide radical, nitric oxide is transformed into peroxynitrite, itself a particularly aggressive agent as an oxidant.

The induction of the synthesis of nitric oxide was shown during the inflammatory response initiated by microbial products during infections or during self-immune reactions. Nitric oxide is therefore an intermediary of physiology which has a positive face by regulating vasodilation and by participating in the immune function, and a negative face when it is emitted in a too large amount.

Present in an excess, it may produce an inflammation, a destruction of cells and of tissues and may also cause vasodilation inducing pain and fatal hypotension during septic or anaphylactic shocks.

It was notably shown that nitric oxide had an influence on the proliferation of keratinocytes (7) and on very strong levels of plasma nitric oxide were observed in subjects having psoriasis (8), suggesting the influence of this intermediary on this disease.

Atopy is a predisposition to the amplified reaction of the immune response. Under its dermatological form, atopic dermatitis affects about 15% of the children of developed countries. It was shown that a high production of nitric oxide is involved in the inflammation, the vasodilation and the oxidative damages to the cells and to the tissues of skins of subjects having atopic dermatitis (9).

Nitric oxide is also involved in the sun's erythema (10).

Also, at the joint level, nitric oxide is involved in inflammatory signs of arthritis. It was also shown in animal models that the administration of NOs inhibitors significantly reduces the inflammation of the cartilage (22).

As regards the systemic inflammatory response to which refers septic shock, it produces a generalized vasorelaxation due to the massive production of nitric oxide, which is expressed by hypotension which may lead to death in the absence of a suitable treatment applied as an emergency (11). This mechanism is also the one which develops during an anaphylactic shock (12).

Accordingly, if it may be sometimes desirable to provide nitric oxide to an organ which is lacking thereof like in the case for example in chronic hypertension associated with sequels of myocardial infarction or for preventing it, in many other cases, it would be necessary to neutralize this same nitric oxide in order to reduce its noxious effects, either in an ambulatory way, like in atopic dermatitis, eczema, psoriasis, coughing, or arthrosis, or in a massive emergency way for treating collapses associated with septic or anaphylactic shock.

The production of nitric oxide may be reduced by inhibiting the nitric oxide synthases (NOs), enzymes which release it from L-arginine, or else by neutralizing nitric oxide chemically or by capturing it with suitable complexing agents.

NOs inhibitors are known, among them derivatives of arginine, like methylated, nitrated or propylated derivatives of this amino acid (13), but their use in systemic therapeutics includes serious drawbacks (14). Indeed, the inhibition of NOs is difficulty controllable and for avoiding an overdosage which may lead to pulmonary hypertension, it is often necessary to administer gaseous nitric oxide via a respiratory route at the same time as the inhibitor of NOs, while monitoring the cardiovascular parameters of the patient. This drawback may be due to the lack of specificity of the inhibitors used towards the different isoforms of NOs, but for the moment, no specific inhibitor has proven its efficiency and its reliability in clinical trials.

NOs inhibitors have been proposed for treating esthetic disorders of the skin (15, 16) but their use is limited by the detrimental secondary effects, such as arterial hypertension, which they may cause and the fact that these inhibitors are all synthetic products while consumers today prefer turning themselves towards substances of natural origin.

There also exist molecules capable of neutralizing nitric oxide such as haemoglobin which is considered as the reference molecule in this field, and diverse synthetic molecules like carboxy-2-phenyl-4,4,5,5-tetramethylimidazoline-1-oxyl-3-oxide (carboxy-PTIO) (17) and ruthenium derivatives (18). The use of all these molecules include drawbacks in particular for therapeutic and cosmetic applications; as an example, the derivatives of ruthenium are considered as potentially toxic and carcinogenic.

Apart from these three groups of molecules, a very large number of other components of plant origin have been proposed as possible agents for neutralization of nitric oxide, like flavanoids and more particularly polyphenols. But the evaluation of their neutralizing effect by the Griess method which measures the NO₂⁻ ion, do not allow evaluation of the actual effect of the tested molecule. Indeed, any antioxidant capable of reducing the concentration of oxygenated radicals in the medium is an inhibitor of the reaction which leads from NO to the ion NO₂⁻, and therefore the Griess method provides a wrong indication of the actual effect of the molecule on its power of neutralizing nitric oxide, since it is impossible to distinguish between the antioxidant power and the neutralizing power towards nitric oxide.

With the purpose of having a real appreciation of the neutralizing effect of compound towards nitric oxide, the Inventors have developed a specific method in vitro which directly evaluates the concentration of nitric oxide in the medium and which measures the neutralization thereof in real time in the presence of the tested molecules; example I describes this method.

For this, the nitric oxide is prepared in a separated container by achieving reduction of sodium nitrite with ferrous sulfate and carry away with a nitrogen gas stream into another container containing the product to be tested in solution in a buffer. In this solution, is placed an amperometric probe which measures in real time the current produced by the presence of nitric oxide in solution.

Once provided with this specific method for evaluating the neutralization of nitric oxide, the Inventors were able to confirm the neutralizing effect of certain molecules known as such, like hemoglobin, but also to discover surprisingly that certain molecules, described as good neutralizers of nitric oxide were in reality not so, once passed to the screening of this specific method, notably flavanoids like quercetin, catechin and luteolin.

They also identified molecules which prove to be very good neutralizers of nitric oxide and which have never been described as such.

Thus, the present invention targets the identification of compounds neutralizing efficiently nitric oxide, which may easily be used in therapeutics or in cosmetics and do not have the drawbacks of those described earlier.

The invention relates to a compound of formula (I):

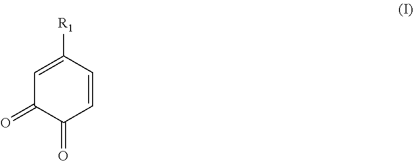

wherein R¹ is selected from the group comprising:
—CH═CH—COOR²

with R² which may represent:
H, a linear C₁-C₃ alkyl chain, optionally substituted with one or several functions selected from among hydroxyl functions and carboxylic acid functions, with a benzene ring, with a diphenol or with a caffeoyl radical;
A saturated or unsaturated C₆ ring, optionally substituted with one or several functions selected from among hydroxyl and carboxylic acid functions and/or with a caffeoyl radical;
The formula Y—O—X-(3,4)diphenol (II) with:
   Y is a pyranose ring, substituted with a CH₂—OH and at least one hydroxyl function and optionally substituted with a rhamnose;
   X may represent the chain CH₂—CH₂— or CH₂—CH(OH)—;
   —Z—O—R³

With:
Z being able to represent the chain CH₂—CH₂—, —CH(OH)—CH₂— or —CH₂—CH(COOH)—;
R³ may represent:
   —CO—CH═CH-(3,4)diphenol or
   The formula W—O—CO—CH═CH-(3,4)diphenol (III) with W being a pyranose ring, substituted with a CH₂—OH and at least one hydroxyl function and optionally substituted with a rhamnose;

for its use for preventing and/or treating diseases and/or disorders resulting from an excess of NO.

By physiologically acceptable, is meant compatible with administration to a subject, preferably a mammal, through any administration route.

The excess of NO may be determined by measuring the level of nitrates in the blood serum (9). It is then considered that a human individual has an excess of NO when its concentration in nitrates in the blood serum is greater than or equal to 14 mmol/L, in particular 30 mmol/L or further 50 mmol/L.

According to an alternative of the invention, the compound of formula (I) is such that R¹ is selected from among the group comprising:
—CH═CH—COOR²

with R² which may represent:
H or
The formula Y—O—X-(3,4)diphenol (II) with:
   Y represents a pyranose ring, substituted with a CH₂—OH and at least one hydroxyl function and substituted with a rhamnose;
   X represents the chain CH₂—CH₂—,
   —Z—O—R³

With:
Z represents the chain CH₂—CH₂—,
R³ represents the formula W—O—CO—CH═CH-(3,4)diphenol (III) with W representing a pyranose ring, substituted with a CH₂—OH and at least a hydroxyl function and substituted with a rhamnose.

According to another alternative of the invention, the latter relates to a compound of formula (Ia) such as:

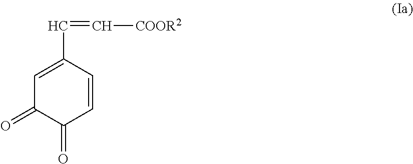

with R² being able to represent:
H, a linear C₁-C₃ alkyl chain, optionally substituted with one or several functions selected from among hydroxyl and carboxylic acid functions, with a benzene ring, with a diphenol or with a caffeoyl radical;
An unsaturated or saturated C₆ ring, optionally substituted with one or several functions selected from the hydroxyl and carboxylic acid functions and/or by a caffeoyl radical;
The formula Y—O—X-(3,4)diphenol (II) with:
   Y is a pyranose ring, substituted with a CH₂—OH and at least one hydroxyl function and optionally substituted with a rhamnose;
   X may represent the chain CH₂—CH₂— or CH₂—CH(OH)—.

According to further other alternatives of the invention, the latter relates to a compound of formula (I) such as:
R¹ is CH═CH═COOR² and R² is an H or a linear C₁ or C₂ alkyl chain; or
R¹ is CH═CH═COOR² and R² fits the formula Y—O—X-(3,4)diphenol (II) with Y being the glucose substituted with a rhamnose and X may represent the chain CH₂—CH₂— or CH₂—CH(OH)—; or
R¹ is Z—O—R³ with Z being able to represent the chain CH₂—CH₂—, —CH(OH)—CH₂— or CH₂—CH(COOH)—; and R³ represents the formula W—O—CO—CH═CH-(3,4)diphenol (Ill) with W being a glucose substituted with a rhamnose.

According to a preferred alternative of the invention, the latter relates to the compounds mentioned below or to their physiologically acceptable salts for their use for preventing and/or treating diseases and/or disorders resulting from an excess of NO:
The quinone of caffeic acid:

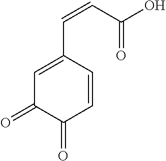 [SMILES: O=C(O)/C=C\C1=CC(=O)C(=O)C=C1]

The quinone of verbascoside has 2 forms:

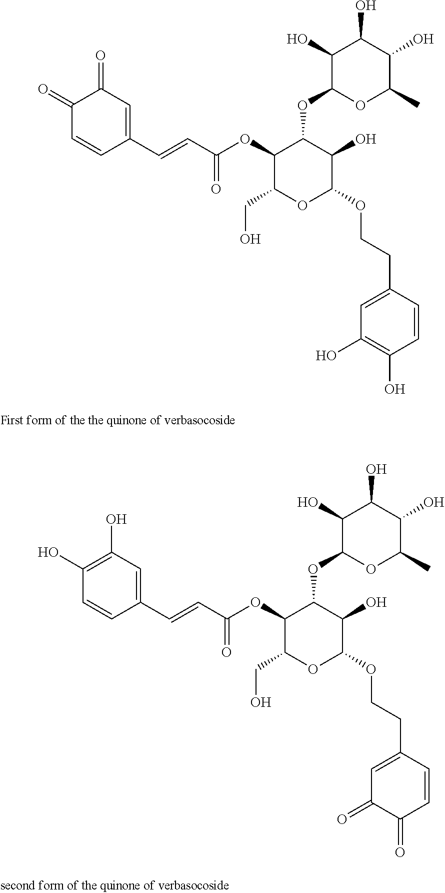 [SMILES: C[C@H]1O[C@@H](O[C@@H]2[C@@H](O)[C@H](OCCc3ccc(O)c(O)c3)O[C@H](CO)[C@H]2OC(=O)/C=C/C2=CC(=O)C(=O)C=C2)[C@@H](O)[C@@H](O)[C@@H]1O ; C[C@H]1O[C@@H](O[C@@H]2[C@@H](O)[C@H](OCCC3=CC(=O)C(=O)C=C3)O[C@H](CO)[C@H]2OC(=O)/C=C/c2ccc(O)c(O)c2)[C@@H](O)[C@@H](O)[C@@H]1O]

By linear, optionally substituted C₁-C₃ alkyl chain, is meant a hydrocarbon chain with 1 to 3 carbon atoms, saturated, linear, optionally substituted such as methyl, ethyl or propyl.

By hydroxyl function is meant the group —OH.

By carboxylic function is meant the group —COOH.

By benzene ring, is meant the aromatic functional group of raw formula C₆H₆:

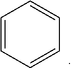

By diphenol, is meant an aromatic compound consisting of a benzene ring and of two hydroxyl functions, of raw formula C₆H₆O₂.

By caffeoyl radical, is meant the radical derived from caffeic acid, of formula: —O—CO—CH═CH-(3,4)diphenol.

By pyranose ring, is meant a saturated C₆ ring, consisting of 5 carbon atoms and one oxygen atom.

As an example, reference may be made of glucose which has a pyranose ring substituted with a —CH₂—OH and 4 OH hydroxyls.

By rhamnose, is meant the ose in its D or L conformation, in its α or β form, of formula:

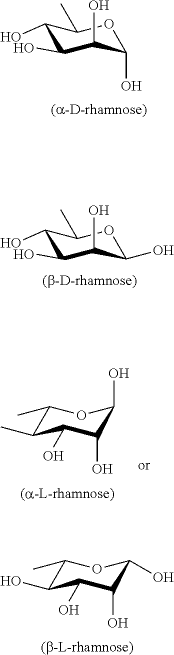

The compounds of formula (I) may be synthesized chemically or else extracted from plants.

As an example, mention may be made of the orthoquinone of caffeic acid which may be obtained in crystallized form, by oxidation of the caffeic acid with o-chloranil, according to a method described in the prior art (20, 21).

Alternatively, the orthoquinone of caffeic acid may be extracted from plants such as Salvia officinalis, Mentha spicata, Cinnamomum verum, Thymus vulgaris.

One skilled in the art knows the methods for extracting chemical compounds from plants. From among these methods, mention may be made of the extraction by means of chemical solvents, with a supercritical fluid such as CO₂, nanofiltration.

The quinone of verbascoside may be extracted from plants from the family of Lamiaceae (Phlomis, Scrophulariaceae, Verbascum phlomoides, Verbascum mallophorum) or from the family of Buddlejaceae (Buddleja globosa, Buddleja cordata) or from the family of Bignoniaceae (Pithecoctenium sp, Tynanthus panurensis) or from the family of Orobanchaceae (Cistanche sp, Orobanche rapumgenistae), from the family of Plantaginaceae (Plantago lanceolata, Verbenaceae, Verbena officinalis, Aloysia citrodora) from the family of Oleaceae (Olea europaea) from the family of Lentibulariaceae (Pinguicula lusitanica) and from the family of Byblidaceae (Byblis liniflora).

Further, the inventors have surprisingly shown and unlike what was assumed in the prior art (19), that an extract of a plant known for its anti-inflammatory effect, plantain (Plantago lanceolata) does not owe its anti-inflammatory effect to phenylhydantoic glycoside, verbascoside which it contains, but to the quinone derived from this polyphenol.

Thus, in another alternative of the invention, the latter relates to an extract of Plantago lanceolata selectively enriched with quinone of verbascoside; such an extract of Plantago lanceolata enriched with verbascoside quinone is preferably obtained from areal parts of the plant by alcoholic maceration; such an extract may be obtained according to the method described in example 5. This particular extract produces a significant effect of neutralization of NO.

The invention relates more particularly to the use of a compound or an extract of Plantago lanceolata according to the invention for preventing and/or treating diseases and/or inflammatory disorders.

In particular, the compounds or the extract of Plantago lanceolata according to the invention are useful for preventing and/or treating skin inflammatory diseases and/or disorders such as psoriasis, atopic dermatitis, contact dermatitis, skin irritation, contact hypersensitivity reaction, skin allergic expressions, excessive vasodilation, rosacea, sun erythema, acne.

The compounds of the extract of Plantago lanceolata according to the invention are further useful for preventing and/or treating joint inflammatory diseases and/or disorders such as arthritis, comprising rheumatoid arthritis, infectious arthritis and osteoarthritis, arthrosis, rheumatoid polyarthritis, ankylosing spondylitis, lupus erythematosus, chondritis.

The compounds or the extract of Plantago lanceolate according to the invention may be used for preventing or treating coughing, whether it is associated with a disease of the respiratory tracts such as bronchial obstruction of the newly born, bronchitis, influenza, whooping cough, tuberculosis, a bronchial cancer or associated with a particular state and/or environment as this is the case of the smoker coughing or caused by passive smoking, allergic coughing, coughing associated with asthma or further with gastro-esophageal reflux.

The compounds or the extract of Plantago lanceolate according to the invention may further be used for preventing or treating cardiovascular collapses associated with a hypovolemic shock, or a cardiogenic shock or to an anaphylactic shock or to a septic shock. Cardiovascular collapse corresponds to a collapse in the blood pressure; the systolic arterial pressure then becomes less than 80 mmHg.

The use of the compounds or of the extract of Plantago lanceolate according to the invention may also have a cosmetic purpose, in order to combat intrinsic or extrinsic ageing of the skin, more particularly for combating signs of cutaneous ageing, such as photo-ageing, wrinkles, fine lines, dried or cracked skins.

From among the other uses of these compounds or of the extract of Plantago lanceolate according to the invention is cosmetics, mention may be made of:
combatting skin neurogenic inflammatory processes;
improving comfort of sensitive skins;
reinforcement of the barrier function of the skin;
stimulation of hydration of the skin;
improving the comfort of dry skins;
controlling sweating;
stimulating lipolysis;
inhibiting hair loss.

The present invention also relates to a pharmaceutical or cosmetic composition containing at least one compound of formula (I) or an extract of Plantago lanceolate according to the invention as well as a physiologically acceptable vehicle.

One skilled in the art will know how to adapt the formulation of the compositions according to the invention according to their physico-chemical properties and to their administration route.

The compositions according to the invention may be administered via any administration route which notably includes the topical route, in particular on the skin or on the hair, but also orally.

Preferably, when the compositions according to the invention are used for treating skin or joint inflammatory diseases and/or disorders, or else when they are used for a cosmetic purpose, they are administered via the topical route and may be selected from among a cream, a gel, an oil, a lotion, a milk.

When the compositions according to the invention are used for treating coughing, preferably they will be administered orally. The selected galenic form will be a syrup, a lozenge to be sucked, a gelatin capsule, a tablet.

### FIGURE

FIG. 1: Apparatus allowing application of the specific method for evaluating the neutralization of nitric oxide as described in example 1.

### EXAMPLES

### Example 1. Method for Evaluating the Neutralization of Nitric Oxide

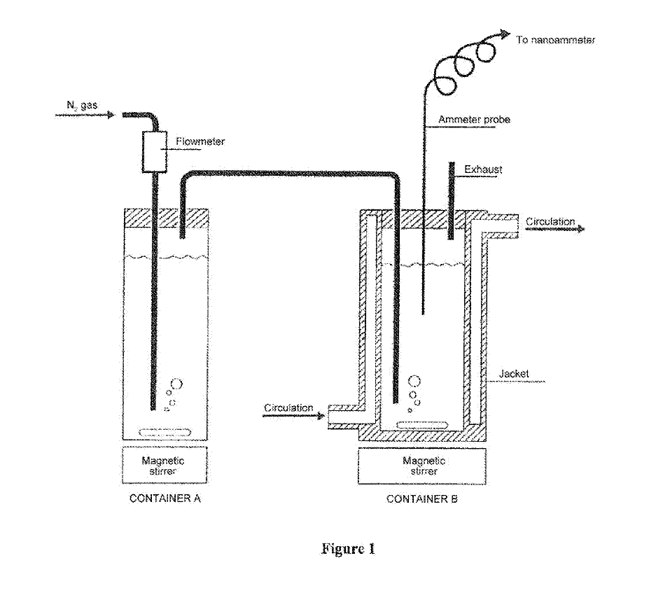

Nitric oxide is prepared extemporaneously in a container A, obturated with a flexible polymeric plug through which pass an entering tubing and an exiting tubing, with an inner diameter comprised between 50 μm and 300 μm, but particularly of 100 μm (cf. FIG. 1), with a volume comprised between 10 ml and 100 ml, but particularly of 30 ml, by adding a volume comprised between 0.1 and 2 ml, but particularly of 0.5 ml of an aqueous solution of sodium nitrite (NaNO₂, ref. Sigma-Aldrich 237213) at a concentration comprised between 0.10% and 2.00% but particularly of 0.70%, at a volume comprised between 5 ml and 80 ml of the following aqueous solution:

| | |
|---|---|
| Sulfuric acid (H₂SO₄, ref. Sigma-Aldrich 32,050-1): | 1 ml |
| Ferrous sulfate (FeSO₄, 7H₂O, ref. Sigma-Aldrich 215422): | 5.5 g |
| Deionized water: | 200 ml |

The container B in which is measured the nitric oxide is a container opaque to light, with a double jacket allowing circulation of water maintaining the temperature at 25° C., obturated with a plug through which pass the tubing stemming from the container A and an exhaust tubing (cf. FIG. 1) with a volume comprised between 10 ml and 100 ml, preferably 30 ml which receives a volume comprised between 5 ml and 80 ml, preferably 20 ml, of an aqueous solution of phosphate buffer at pH 7.4.

Once they are mixed, the solutions are stirred for the whole period of the measurement with a magnetic stirrer, in container A and in container B.

Nitrogen gas is admitted at a flow rate comprised between 20 ml/min and 120 ml/min, preferably 80 ml/min in the bottom of the container A. The nitrogen thereby carries away the nitric oxide through the exiting tubing of container A as far as into container B where it is measured with a device provided for this purpose equipped with an amperometric probe provided with a specific membrane (apparatus of ref. TBR 1025 from World Precision Instruments, probe ISO-NOP). The probe thereby delivers a variable current between 10 and 1,000 nA proportional to the concentration of nitric oxide in the tested solution, of the order of magnitude of μM. The electric current produced by the probe under the influence of nitric oxide is recorded with a suitable device.

Before any measurement, the probe is calibrated by measuring the current produced by releasing known increasing amounts of nitric oxide. It is thus checked that there exists a linear relationship between the electric current produced by the probe and the molar concentration of nitric oxide and the correspondence between this electric current and the molar concentration of nitric oxide is determined. For this, 20 ml of the following aqueous solution is placed in the container B disconnected from the container A during the calibration period:

| | |
|---|---|
| Sulfuric acid (H₂SO₄, ref. Sigma-Aldrich 32,050-1): | 1 ml |
| Potassium iodide (KI, ref. Sigma-Aldrich 12636): | 3.31 g |
| Deionized water: | 200 ml |

50 μl, 100 μl, 150 μl and 200 μl of a solution of sodium nitrite (NaNO₂, ref. Sigma-Aldrich 237213) are then successively added to a concentration of 360 μM.

The curve giving the correspondence between the nitric oxide concentration in the reaction medium and the electric intensity produced by the probe expressed in nA is plotted for each addition of the nitric oxide solution.

### Evaluation of the Neutralization Effect on a Molecule to be Tested

During a typical measurement, 20 ml of the ferrous sulfate solution are placed in the container A and 20 ml of phosphate buffer at pH 7.4 are placed in the container B. In the container A, nitrogen gas is admitted for 10 mins, which passes in transit towards the container B, in order to remove the dissolved oxygen present in the solutions of the containers A and B. After these 10 mins, the recording of the measurement of nitric oxide is launched.

At time T₀, the sodium nitrite solution is injected into the container A through the flexible polymeric plug by means of a syringe equipped with a needle. The recording of the nitric oxide shows a positive evolution which will stabilize shortly after about 3 mins, before slowly decreasing in order to return to the baseline in about 20 mins. At time T₁=T₀+4 mins, 400 μl of a hydro-ethanol solution with an alcoholic titer comprised between 0% and 100% according to the solubility of the product to be tested, are injected, containing an amount P of product from 4 to 8 mg, according to the efficiency of the product to be tested, through the flexible plug of the container B by means of a syringe equipped with a needle.

A control is produced with 400 μl of solvent in which is dissolved the product to be tested.

The value E of the neutralization effect of nitric oxide by the product to be tested, expressed in μM of nitric oxide neutralized per second and per mg of product to be tested is obtained by the following expression:

$$E = \frac{Δ {H}_{test} - Δ {H}_{tem}}{30 P} \qquad ( 1 )$$

With: ΔHₜₑₛₜ=difference in concentration in nitric oxide test, expressed in pM of nitric acid between T₁ and T₂=T₁+30 s, obtained with the solution of product to be tested. ΔHₜₑₘ=difference in concentration of control nitric oxide, expressed in pM of nitric oxide between T₁ and T₂=T, +30 s, obtained with the solvent of the product to be tested.
P=amount of product to be tested in mg.

### Example 2. Measurement of the Neutralization Effect of Nitric Oxide and Effects of Diverse Molecules

The test described in example 1 was applied for testing the following molecules:

Rosmarinic acid (Aldrich 536954)
p-coumaric acid (Sigma C9008)
(+) catechin hydrate (Sigma C1251)
Caffeic acid (Sigma-Aldrich C0625)
Chlorogenic acid (Sigma-Aldrich C3878)
Quercetin dihydrate (Sigma Q0125)
Porcine hemoglobin (Sigma H4131)

### Verbascoside (HWI Analytik GMGH 0082-05-80)

### Luteolin (Sigma L9283).

After having calibrated the probe for measurement of the concentration of nitric oxide of the apparatus described in example 1, in the container A, as described in example 1 and illustrated in FIG. 1, 20 ml of an aqueous solution is placed:

| | |
|---|---|
| of sulfuric acid (H₂SO₄, ref. Sigma-Aldrich 32,050-1): | 1 ml |
| of ferrous sulfate (FeSO₄, 7H₂O, ref. Sigma-Aldrich 215422): | 5.5 g |
| of deionized water: | 200 ml |

In the container B, as described above and in FIG. 1, 20 ml of a phosphate buffer solution at a pH of 7.4 is placed.

Nitrogen gas is admitted into the apparatus at a flow rate of 80 ml/min for 10 mins, and then the recording of the value of the intensity of the electric current produced by the probe for detection of nitric oxide is launched and (at time T₀) it is injected into the container A, a 0.5 ml of an aqueous solution of sodium nitrite (NaNO₂, ref. Sigma-Aldrich 237213) at a concentration of 0.70%.

At time T₁=T₀+4 mins, 400 μl of a hydro-ethanol solution with an alcoholic titre comprised between 0% and 100% depending on the solubility of the product to be tested, containing an amount P of product to be tested expressed in mg, adjusted according to the efficiency of the tested product.

The values of the concentrations of nitric oxide are noted at time T₀, T₁=T₀+4 mins and T₂=T₁+30 s. The values of E calculated according to formula (1) appears in the table 1 below.

TABLE 1

| Tested molecules | E (μM · s⁻¹· mg⁻¹⁾ |
|---|---|
| Hemoglobin (control+) | 2.27 |
| Caffeic acid | 0.957 |
| Rosmarinic acid | 0.043 |
| p-coumaric acid | 0.13 |
| Catechin | 0.13 |
| Chlorogenic acid | 0.043 |
| Luteolin | 0.174 |
| Quercetin | 0.087 |
| Verbascoside | 0.058 |

### Example 3. Preparation and Measurement of the Effect of o-Quinone of Caffeic Acid

The ortho-quinone of caffeic acid:

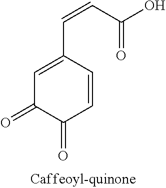 [SMILES: O=C(O)/C=C\C1=CC(=O)C(=O)C=C1]

may be obtained crystallized, by oxidation of caffeic acid by the o-chloranil in solution in a mixture of ether and of tetrahydrofurane (4/1) at −70° C., according to a method described in the prior art (20, 21).

According to the method for measuring the neutralization effect of nitric oxide as described in example 1, the caffeoyl-quinone produces an effect E of 24.36 μM·s⁻¹·mg⁻¹.

A high value of E is therefore observed for this molecule comparatively with the effects produced by molecules already known as capable of neutralizing nitric oxide.

### Example 4. Preparation of an Extract of Plantain and Evaluation of its Neutralization Activity of Nitric Oxide

In order to determine the method for treating the plant and the extraction method leading to better neutralization efficiency of nitric oxide, it was proceeded with the extraction of a dry plant and of a fresh plant with hydro-alcoholic solutions with titers varying from 10 to 10 ranging from 0% to 100% of ethanol, either by maceration at room temperature for 48 h, in solvent volumes corresponding to 10 times the weight of the plant (v/w), or by extraction upon boiling at reflux for 30 mins with solvent volumes also corresponding to 10 times the weight of the plant (v/w). Subsequently to these preliminary tests, the following method was selected, since it produces the most active extract:

Aerial parts of Plantago lanceolate are harvested and rapidly frozen at −18° C. in order to ensure their preservation. 1 kg of this batch is coarsely milled and put to ripen in 10 liters of aqueous ethanol at 60% for 48 h. This alcoholic titer is defined by taking into account the water content of the plant, determined beforehand by drying on a representative sample. At the end of the maceration, the plant is removed by sifting, and then the extracted solution is filtered on a filter with 0.45 μM of porosity for removing the major portion of the germs present. The solution is then concentrated under reduced pressure so as to obtain a total volume of 1 l, totally without any ethanol.

This solution is then dried by freeze-drying in order to obtain a final weight of 43 g.

The efficiency E of neutralization of nitric oxide is 0.16 μM·s⁻¹·mg⁻¹.

### Example 5. Preparation of an Extract of Aerial Parts of Plantago lanceolate Enriched with o-Quinones from Verbascoside

Aerial parts of Plantago lanceolate are harvested and rapidly frozen to −18° C. in order to ensure their preservation. 1 kg of this batch is coarsely milled and set to ripen in 10 liters of aqueous ethanol at 60% for 48 h. This alcoholic titer is defined by taking into account the water content of the plant, determined beforehand by drying on a representative sample. At the end of the maceration, the plant is removed by sifting, and then the extracted solution is filtered on a filter with 0.45 μM of porosity for removing the major portion of the germs present. The solution is then concentrated under reduced pressure so as to obtain a total volume of 1 l, totally without any ethanol.

This concentrated solution is then extracted with a counter current with 5 times 200 ml of ethyl acetate. The organic solutions are collected, dried on anhydrous sodium sulfate, filtered and then dry evaporated under reduced pressure. 1.12 g of dry extract is thereby obtained which is taken up with 25 ml of pure ethanol.

This ethanol solution is subject to flash chromatography (apparatus Grace Reveleris X2) on a silica gel column of 40 g (Grace Reveleris Silica Cartridge 40 g) with a dichloromethane-methanol gradient like in table 2 below:

TABLE 2

| Time (mins) | Dichloromethane % | Methanol % |
|---|---|---|
| 0 | 100 | 0 |
| 15 | 80 | 20 |
| 18 | 80 | 20 |
| 18.5 | 50 | 50 |
| 23.5 | 50 | 50 |

The fractions of 2 ml which are subject to the neutralization test of nitric oxide are collected. The most active fractions are retained which correspond to the elution volumes of 568 ml to 576 ml, which provide the whole 17.7 mg of dry material after evaporation of the solvent under reduced pressure, and which has a neutralization efficiency E of nitric oxide of 0.96 μM·s⁻¹·mg⁻¹.

The presence of the o-quinone of the verbascoside is then checked in high resolution mass spectrometry in tandem, with electrospray ionization in a negative mode, by the presence of a doubly charged ion at m/z 311, corresponding to the relevant quinone and having the characteristic fragmentation of phenylhydantoic heterosides, in this case related to that of the verbascoside.

### Example 6. Galenic Formula of a Cream Containing an Extract of Plantain, Intended for Treating Skin Inflammations (Notably Dermatitises, Eczema, Psoriasis) Adapted to Body Use

A cream is prepared having the following weight formula, according to a method and with standard materials as known from the prior art:

| | |
|---|---|
| Purified water: | 67.28% |
| Glycerol (Glycerine, AMI Chimie): | 20% |
| Cetostearyl alcohol (Lanette O, AMI Chimie): | 7% |
| Palmitostearic acid (Stearin, Stearinerie Dubois): | 3% |
| Extract of fresh aerial parts | 1% |
| of Plantago lanceolata as described in example 5: | |
| Benzyl alcohol (Geogard 221, Lonza): | 0.87% |
| Cetostearyl sodium sulfate (Lanette E, AMI Chimie): | 0.7% |
| Dehydroacetic acid (Geogard 221, Lonza): | 0.09% |
| Sodium hydroxide (Sigma-Aldrich): | 0.058% |

### Example 7. Galenic Formula of a Cream Containing a Plantain Extract, Intended for Treating Skin Inflammations (Notably Dermatitises, Eczema, Psoriasis) Adapted for Use on the Face

| | |
|---|---|
| Purified water: | 67.28% |
| Glycerol (Glycerine, AMI Chimie): | 10% |
| Cetostearyl alcohol (Lanette O, AMI Chimie): | 7% |
| Isopropyl myristate (Isopropyl Myristate, Interchimie): | 3% |
| Extract of fresh aerial parts | 1% |
| of Plantago lanceolata as described in example 5: | |
| Benzyl alcohol (Geogard, Lonza): | 0.87% |
| Cetostearyl sodium sulfate (Lanette E, AMI Chimie): | 0.7% |
| Dehydroacetic acid (Geogard, Lonza): | 0.09% |
| Sodium hydroxide (Sigma-Aldrich): | 0.058% |

### Example 8. Galenic Formula of a Washing Gel Containing a Plantain Extract, Intended for Cleaning Hair, the Face and the Body of Subjects Having Skin Inflammations (Notably Dermatitises, Eczema, Psoriasis)

| | |
|---|---|
| Mixture of fatty alcohol ethersulfate (Texapon ASV50, AMI | 50% |
| Chimie): | |
| Purified water: | 34.03% |
| Glycerol (Glycerine, AMI Chimie): | 10% |
| Sodium chloride (sodium chloride, VWR): | 4% |
| Extract of fresh aerial parts | 1% |
| of Plantago lanceolata as described in example 5: | |
| Benzyl alcohol (Geogard 221, Lonza): | 0.87% |
| Dehydroacetic acid (Geogard 221, Lonza): | 0.09% |
| Sodium hydroxide (Sigma-Aldrich): | 0.01% |

### Example 9. Galenic Formula of a Gel Intended for Treating Skin Inflammations (Notably Dermatitises, Eczema, Psoriasis) Adapted to Body Use and to Use on the Face

| | |
|---|---|
| Purified water: | 95.69% |
| Carbomer (Carbopol 980, Lubrizol): | 2% |
| Extract of fresh aerial parts | 1% |
| of Plantago lanceolata as described in example 5: | |
| Benzyl alcohol (Geogard 221, Lonza): | 0.87% |
| Dehydroacetic acid (Geogard 221, Lonza): | 0.09% |
| Sodium hydroxide (Sigma-Aldrich): | 0.35% |

### Example 10. Galenic Formula of a Cream Containing a Plantain Extract, Intended for Treating Inflammations Relative to Acne

| | |
|---|---|
| Purified water: | 71.04% |
| Cetostearyl alcohol (Lanette O, AMI Chimie): | 10% |
| Essential oil of lemon (Myrtea): | 7.5% |
| Essential oil of sweet orange (Myrtea): | 7.5% |
| Extract of fresh aerial parts | 1% |
| of Plantago lanceolata as described in example 5: | |
| Cetostearyl sodium sulfate (Lanette E, AMI Chimie): | 2% |
| Benzyl alcohol (Geogard 221, Lonza): | 0.87% |
| Dehydroacetic acid (Geogard 221, Lonza): | 0.09% |

### Example 11. Galenic Formula of a Syrup for Coughs

| | |
|---|---|
| Purified water: | 44.23% |
| Saccharose: | 44.22% |
| Glycerol (Glycerine, AMI Chimie): | 5% |
| Extract of fresh aerial parts | 4% |
| of Plantago lanceolata as described in example 5: | |
| Natural raspberry aroma: | 1.5% |
| Citric acid monohydrate: | 0.75% |
| Sodium benzoate: | 0.3% |

### Example 12. Galenic Formula of Gelatin Capsules for Treating Inflammatory Signs of Arthrosis

For a gelatin capsule no. 0 (Capsugel):

| | |
|---|---|
| Extract of fresh aerial parts | 0.350 g |
| of Plantago lanceolata as described in example 5: | |
| Maltodextrin: | 0.045 g |

### REFERENCES

1. Vascular endothelial cells synthesize nitric oxide from L-arginine. R. M. Palmer, D. S. Ashton, S. Moncada. Nature, Vol. 333, 6174: 664-666.
2. Nitric oxide release accounts for the biological activity of endothelium-derived relaxing factor. R. M. Palmer, A. G. Ferrige, S. Moncada. Nature, Vol. 327, 6122: 524-526.
3. Macrophage Oxidation of L-Arginine to Nitrite and Nitrate: Nitric Oxide is an intermediate. M. A. Marietta, P. S. Yoon, R. Iyengar, C. D. Leaf, J. S. Wishnok. Biochemistry, 1988, 27:8706-8711.
4. Nitric oxide: a cytotoxic activated macrophage effector molecule. J. B. Hibbs, R. R. Taintor, Z. Vavrin, E. M. Rachlin. Biochemical and Biophysical Communications, 1988, Vol. 157,1:87-94.
5. Biosynthesis of nitric oxide from L-Arginine. A pathway for the regulation of cell function and communication. S. Moncada, R. M. J. Palmer, E. A. Higgs. Biochemical Pharmacology, 1989, Vol. 38, 11: 1709-1715.
6. Modulation of acute inflammation by endogenous nitric oxide. A. Ialenti, A. Ianaro, S. Moncada, M. Di Rosa. European Journal of Pharmacology, 1992, Vol. 211:177-182.
7. Nitric oxide and its implication in skin homeostasis and disease A review. D. Bruch-Gerharz, T. Ruzicka, V. Kolb-Bachofen. Arch. Dermatol. Res., 1998, 290:643-651.
8. Nitric Oxide Levels in Patients with Psoriasis Treated with Methotrexate. N. S. Tekin, N. Iiter, B. Sancak, M. G. Ozden, M. A. Gurer. Mediators of Inflammation, 2006, Vol. 2006: 1-5.
9. New markers of disease activity in children with atopic dermatitis. A. H. A. Mohsen, H. A. Wahab, E. Allam. Journal of American Science, 2011, Vol. 7(10): 404-408.
10. Nitric oxide function in the skin. M. M. Cals-Grierson, A. D. Ormerod. Nitric Oxide, 2004, Vol. 10:179-193.
11. The role of nitric oxide in sepsis an overview. K. A. Kirkeben, A. Strand. Acta Anaesthesiol. Scand., 1999, Vol. 43: 275-288.
12. Role of Nitric Oxide in Anaphylactic Shock. H. Mitsuhata, R. Shimizu, M. M. Yokoyama. Journal of Clinical Immunology, 1995, Vol. 15(6).
13. Arginine-Based Inhibitors of Nitric Oxide Synthase: Therapeutic Potential and Challenges. J. Vitecek, A. Lojek, G. Valacchi, L. Kubala. Mediators of Inflammation, 2012, Vol. 2012: 1-22.
14. Inhibition of nitric oxide synthase during sepsis: revival because of isoform selectivity, W. Stahl, M. Matejovic, P. Radermacher. Shock, 2010, Vol. 34, (3): 321-322.
15. Nos inhibitors for treatment of wrinkles. S. Fujii, E. Lerner. Brevet PCT/US2002/002292.
16. Inhibitors of NO synthase and uses. M. M. Cals-Grierson. Brevet PCT/FR2002/002064.
17. Beneficial effect of carboxy-PTIO on hemodynamic and blood gas changes in septic shock dogs. Chieko Mitaka, Yukio Hirata, Kuninori Yokoyama, Takashi Nagura, Yukio Tsunoda and Keisuke Amaha. Crit Care 1997, 1:45.
18. Ruthenium complexes as nitric oxide scavengers: a potential therapeutic approach to nitric oxide-mediated diseases. Fricker S P, Slade E, Powell N A, Vaughan O J, Henderson G R, Murrer B A, Megson I L, Bisland S K, Flitney F W. Br. J. Pharmacol. 1997, 122(7):1441-9.
19. Assessment report on Plantago lanceolate L., folium. Collective work. European Medicines Agency. EMA/HMPC/437859/2010. Committee on Herbal Medicinal Products (HMPC).
20. The synthesis and isolation of caffeoquinone and caffeoquinone methyl ester. Davies R. Tetrahedron letters, 1976, 4: 313-314.
21. Oxidation Products of Caffeic Acid as Model Substances for the Antigonadotropic Activity of Plant Extracts. Mathias John, Hans Gerd Gumbinger, and Hilke Winterhoff. Planta Medica, 1990, 56:14-18.
22. Nitric oxide in arthritis. Daniel Jang and George A. C. Murrell. Free Radical Biology & Medicine, 1998, 24(9): 1511-1519.

1. A compound of formula (I): 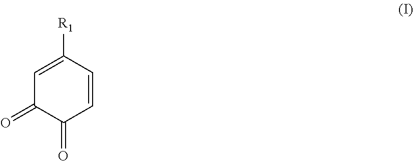
for preventing and/or treating diseases and/or disorders resulting from an excess of NO, wherein:
R¹ is selected from the group consisting of —CH═CH—COOR² and —Z—O—R³;
is a group of formula —Y—O—X-(3,4)diphenol (II);
is a pyranose ring, substituted with —CH₂—OH, at least one hydroxyl function and a rhamnose;
and Z are —CH₂—CH₂—;
R³ is a group of formula —W—O—CO—CH═CH-(3,4)diphenol (III); and
W is a pyranose ring, substituted with —CH₂—OH, at least one hydroxyl function and a rhamnose;

2. The compound according to claim 1 selected from the group consisting of a first form of the quinone of the verbascoside and a second form of the quinone of the verbascoside.

3. An extract of Plantago lanceolata enriched with a quinone from verbascoside.

4. An extract of Plantago lanceolata enriched with a quinone from verbascoside, where the extract is obtained from aerial parts of the plant by alcoholic maceration.

5. The compound according to claim 1 for preventing and/or treating inflammatory diseases and/or disorders.

6. The compound according to claim 1 for preventing and/or treating skin inflammatory diseases and/or disorders selected from the group consisting of psoriasis, atopic dermatitis, contact dermatitis, skin irritation, contact hypersensitivity reaction, skin allergic signs, excessive vasodilation, rosacea, solar erythema and acne.

7. The compound according to claim 1 for preventing and/or treating joint inflammatory diseases and/or disorders selected from the group consisting of arthritis, arthrosis, rheumatoid polyarthritis, ankylosing spondylarthritis, erythematous lupus and chondritis.

8. The compound according to claim 1 for preventing and/or treating coughs.

9. The compound according to claim 1 for preventing and/or treating anaphylactic shocks or septic shocks.

10. The compound according to claim 1 for combatting intrinsic or extrinsic ageing of the skin; for combatting signs of skin ageing; for combatting skin neurogenic inflammatory processes; for improving the comfort of sensitive skins; for reinforcing the barrier function of the skin; for stimulating hydration of the skin; for improving the comfort of dry skins; for controlling sweating; for stimulating lipolysis; and for inhibiting hair loss.

11. A pharmaceutical or cosmetic composition containing at least one compound of formula (I) according to claim 1 and a physiologically acceptable carrier.

12. The composition according to claim 11, for application on skin or for oral administration.

13. The composition according to claim 12, wherein the composition is a cream, a gel, an oil, a lotion or a milk.

14. The composition according to claim 12, wherein the composition is a syrup, a lozenge to be sucked, a gelatin capsule or a tablet.

15. A method for preventing and/or treating diseases and/or disorders resulting from an excess of NO, comprising administering to a mammal in need thereof, a therapeutic amount of the compound according to claim 1.

16. The method according to claim 15, wherein the compound is applied to skin or administered orally.

17. A method for preventing and/or treating diseases and/or disorders resulting from an excess of NO, comprising administering to a mammal in need thereof, a therapeutic amount of the composition according to claim 11.

18. The method according to claim 17, wherein the composition is applied to skin or administered orally.
